(19) United States
(12) Patent Application Publication
(10) Pub. No.: US 2019/0145966 A1
(43) Pub. Date: May 16, 2019

(54) MEMBRANE-BASED ANALYTICAL DEVICE FOR BODILY FLUIDS

(71) Applicant: MEJE AB, Uppsala (SE)

(72) Inventor: Per Ove ÖHMAN, Uppsala (SE)

(73) Assignee: MEJE AB, Uppsala (SE)

(21) Appl. No.: 16/092,960

(22) PCT Filed: Apr. 11, 2017
(86) PCT No.: PCT/SE2017/050355

§ 371 Date: Oct. 11, 2018

(30) Foreign Application Priority Data
Apr. 12, 2016 (SE) 1650489-6

(51) Int. Cl. G01N 33/537; C12Q 1/6813; G01N 33/543

(52) U.S. Cl. CPC G01N 33/537; G01N 33/558; G01N 33/54386; C12Q 1/6813

(57) ABSTRACT

Disclosed is an analytical device including at least one non-binding membrane; at least one liquid sample application region; and at least one capturing chamber including labelled particles; in which device the sample application region(s) are arranged upstream of the membrane while the capturing chamber(s) are arranged downstream of the membrane, and wherein the cut-off of the membrane is large enough to allow passage of labelled particles but small enough to retain clusters including analyte bound to particles. Also disclosed is a method of detecting one or more analytes such as biomarkers in a liquid sample such as blood using a device as disclosed.

### TECHNICAL FIELD

The present invention relates to the area of healthcare, and more specifically to fast and simple analysis of liquid samples, such as blood samples, using an analytical device including a non-binding membrane arranged to include with labelled particles capable of recognising a desired analyte.

### BACKGROUND

Healthcare is the maintenance or improvement of health via the diagnosis, treatment, and prevention of disease. In order to identify disease or other health conditions, assays are frequently used. An assay is an analytic procedure for measuring the presence or amount or the functional activity of an analyte, such as a protein or genetical information e.g. DNA, RNA or SNPs. Generally, assays involve biological material or phenomena, which are intrinsically complex, either in composition or in behaviour or both. Advanced assays have become a routine in most healthcare laboratories, often with automated organisation of the procedure from the ordering an assay to pre-analytic sample processing such as sample collection, necessary manipulations e.g. spinning for separation or other processes, aliquoting if necessary, storage, retrieval, pipetting/aspiration etc.

In contrast to the above, point-of-care testing or bedside testing is defined as medical diagnostic testing at or near the point of care i.e. at the time and place of patient care. Usually, simplicity is not achievable until technology develops not only to make a test possible at all but then also to mask its complexity. For example, various kinds of urine test strips have been available for decades, while portable ultrasonic devices became widespread more recently.

Home tests allow testing for some diseases or conditions at home and are known to be cost-effective, quick, and confidential. Home tests can be grouped into three categories: (i) Detection of possible health conditions when no symptoms appear. Such tests will enable early treatment and may also reduce the risks of developing later complications. Examples in this category are cholesterol and hepatitis testing, (ii) Detection of specific conditions when no symptoms appear. Such tests may lead to immediate action. An example in this category is pregnancy testing, and (iii) Monitoring of conditions to allow frequent changes in treatment, such as glucose testing, to monitor blood sugar levels in diabetes.

Some home tests are best evaluated together with the relevant medical history, a physical exam, and other testing; while some are more related to general well-being.

In fact, wellness monitoring is rapidly increasing in today's society, where the wellbeing of the individual is a high priority and enabling technology is becoming readily accessible. “Worried well” is a growing and already large group of people, with a desire and demand for improved home tests and simple and user-friendly devices.

US 2006/0257854 (McDevitt et al) relates to an analyte detection device and a method related to a portable instrument suitable for point-of-care analyses. The device is capable of obtaining diagnostic information using cellular-based analyses and may be used in conjunction with membrane- and/or particle based analysis cartridges. The intended analytes include proteins, cells and microbes.

Further, WO2010/124001 (Advandx Inc) relates to analysis of cells and other analytes, such as biomolecules. More specifically, an objective of WO 2010/124001 is to provide improved methods for the analysis of pathogenic organisms. This is obtained by a device, into which a sample is inserted and divided between a number of channels each one of which is in contact with a reservoir containing reagents for analysing cells, particles or analytes bound to particles. Each channel is also in contact with a membrane, the cut-off of which prevents cells or particles from passing, which membrane is located so that a fluid sample reaches the reservoir before reaching the membrane. Thus, the membrane will capture the analytes where they may be analysed by detection, enumeration and/or identification. The reagents for analysing may include a plurality of reagents that are optically distinguishable and that bind to different cells, particles or analytes resulting in a multiplex device with proposed use for decision making or point-of-care.

WO 2009/014787 (Nanogen Inc) relates to compositions, systems and methods for detecting multiple analytes from a sample. More specifically, a method of assaying a sample is described, which includes to provide a sample to a sample collection device (SCD) comprising a detecting probe and a capture probe, both of which are capable of specifically binding an analyte; and administering a sample from the SCD to a test device (TD) for detection of one or more analytes. The TD may comprise a lateral flow membrane in a body, and a chamber upstream of the lateral flow membrane containing a fluid or solution.

Currently, an increased focus is on preventive measures and healthy living and there is an ongoing demand in this field for improved methods and devices.

### SUMMARY OF THE INVENTION

One object of the present invention is to provide an alternative assay for biomarkers and/or health metrics, which is simple for the user.

Another objective is to provide a device suitable for home testing.

A further objective is to provide an assay, which provides for bespoke solutions for high sensitivity and/or high reproducibility analyses of one or more analytes.

These and other objectives may be achieved by a device as described in the appended claims.

Further detail, embodiments and advantages will appear from the application as a whole including drawings and the description below.

### DESCRIPTION OF DRAWING

FIG. 1 shows schematically a cross section of an illustrative device according to the invention, illustrating how a non-binding membrane divides the tube horizontally. More specifically, FIG. 1 shows a non-binding membrane 1, the sample side 2 of the membrane 1; an sample here illustrated as a blood drop 2′: a capturing chamber 3; particles 4; an optional inlet for wash liquid 5; and a detector 6. A capturing according to the invention may be enclosed by a material 5′.

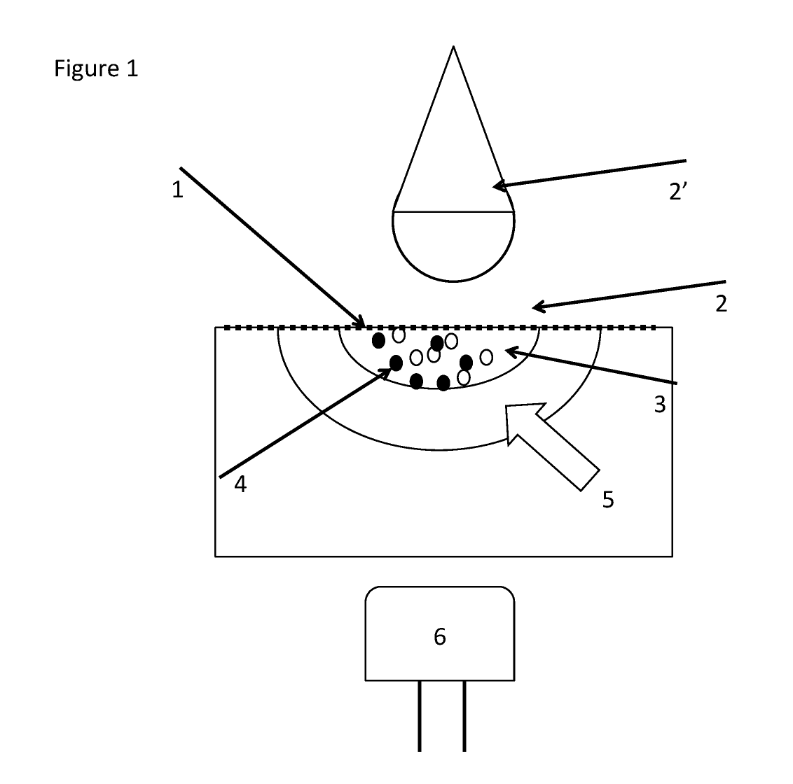

FIG. 2 shows an example of how a number of capturing chambers 3 may be arranged in a tube 8 including a non-binding membrane 1, wherein the sample injection 7 is shown to the left.

### DETAILED DESCRIPTION OF THE INVENTION

A first aspect of the invention is an analytical device comprising at least one non-binding membrane; at least one liquid sample application region; and at least one capturing chamber comprising labelled particles; in which device the sample application region(s) are arranged upstream of the membrane while the capturing chamber(s) are arranged downstream of the membrane, and wherein the cut-off of the membrane is large enough to allow passage of labelled particles but small enough to retain clusters comprised of analyte bound to particles. As the skilled person will appreciate, the amount of particles should be adapted to the kind of sample, the flow and other relevant conditions.

The membrane arranged in the device according to the invention should be substantially unreactive towards the analyte and other components of the liquid sample. Thus, it is non-binding. In this context, the term “non-binding” means that the membrane operates by separating molecules based on size rather than chemical properties.

In one embodiment, the membrane has a non-deformable pore structure with no lateral crossovers between individual pores.

In an advantageous embodiment, the capturing chamber(s) have been arranged in a material from which air has been evacuated and air is prevented to re-enter. For example, the chamber(s) may be surrounded by a porous material such as silicone of a suitable quality, which is easily evacuated by suction of any air by conventional means.

In order to prevent air from re-entering the material, a tape or other air-tight arrangement is provided, which is easily removed. This material may be used to drive a liquid across the non-binding membrane, simply by removing the tape.

The membrane may advantageously be substantially inert and optionally transparent. As the skilled person will appreciate, a membrane may be rendered optically transparent after completed filtration by adding a pore filling material having suitable optical properties, whereby visual inspection is enabled. In one embodiment of the device, the membrane is composed of a high purity metal oxide, such alumina. In a specific embodiment, the membrane is a Whatman™ Anodisc inorganic membrane, or an Anopore membrane (available from www.gelifesciences.com).

The purpose of the particles of the present device is to provide for binding of analyte. Particles according to the invention may for example be proteins or peptides including one or more epitopes capable of binding analyte(s); or nucleic acid molecules wherein the desired sequence capable of binding analyte(s) has been included. In an advantageous embodiment, the particles are comprised of DNA.

In yet another embodiment, the particles are comprised of organic or inorganic carriers, to which receptor(s) specific to the analyte have been attached. The carriers may be of any commonly used kind, and may be porous or non-porous. In one embodiment, the carriers are made from synthetic polymers, such as polystyrene, or copolymers, such as styrene/DVB. In a specific embodiment, the carrier is a dendrimer.

Receptors for coupling to the carrier may be any commonly used binding entity. For example, receptors may be affinity molecule(s), which are commonly proteins, capable of capturing an analyte using a ‘lock/key’ kind of interaction including chemical binding as well as steric interaction. Alternatively, suitable receptors are antibodies, such as monoclonal antibodies, antibody fragments or fusion proteins comprising antibodies or fragments thereof. Further, the receptors may be, or include, suitable nucleic acids, such as DNA, which are capable of capturing an analyte. Suitable antibodies and/or DNA capable of targeting analyte may be prepared using standard techniques. In this context, the skilled person will appreciate that the particles direct or indirect capturing of analyte(s) of interest may be based on any one of many possible ways of interaction, as exemplified above, and the invention in its broad sense is not limited to any specific capturing mechanism.

Thus, the particles provided in the device according to the invention should be capable of capturing an analyte of interest, whereby clusters of analyte with one or more particles are formed. The size of the clusters should be above the cut-off of the membrane, to keep the analyte within the capturing chamber during wash and subsequent detection.

To enable removal of unbound particles, the size of the particles should be selected to be below the cut-off of the membrane, as will be discussed in more detail below.

In an advantageous embodiment, two differently labelled particles directed to unique and different binding sites of the same analyte are arranged in each capturing chamber. For example, if the particles are proteins, each protein will include more than one epitope capable of recognising the analyte of interest.

The skilled person will appreciate how the device according to the invention may be varied into a multiplex device by adding one or more additional capturing chambers comprising particles capable of specific binding of different analytes.

In one embodiment, the capturing chamber(s) are arranged in a tube, such as a capillary.

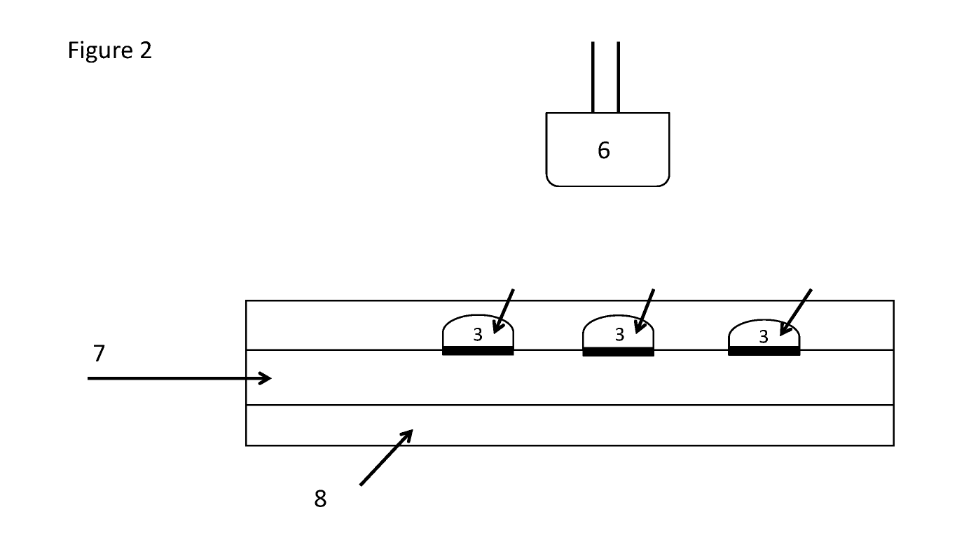

FIG. 2 illustrates a specific embodiment of the invention which includes such a tube. When the device is used, a sample is first provided to a region of the device, such as by injection into the device, e.g. as a blood sample is obtained from a human using a needle of commonly used dimensions. If the tube is of suitable dimensions, the sample may be drawn into the device using capillary forces. The analyte will be driven across the membrane by a suitable force, such as vacuum, as discussed above. As the skilled person will understand, if a biological sample is used, the analyte will be accompanied by a number of other, smaller or similarly sized molecules as it enters the capturing chamber 3. Such other molecules or materials may be e.g. proteins, peptides, DNA, RNA, fat components, cells debris etc. Depending on the nature of the sample and the purpose of the detection, the clusters formed between particles 4 and analyte may be washed by flushing a suitable wash liquid across the membrane 1 using wash liquid inlet 5, resulting in a cleaned capturing chamber and more efficiently detected clusters. In such a wash, any unbound labelled particles should advantageously also be removed from the capturing chamber. Thus, in one embodiment, the cut-off of the membrane is large enough to allow passage of labelled particles but small enough to retain clusters comprised of analyte bound by particles in the capturing chamber. The skilled person in this area may choose suitable buffers or other wash liquids based on their common general knowledge.

The tube or capillary used in the present device may be of a material suitable for management of biological fluids, such as blood, plasma, saliva, sweat or urine. In a preferred embodiment, the tube is of dimensions enabling the transport of a small volume of sample from the extraction point and into the capturing chamber.

The tube material should be sufficiently transparent to gas such as oxygen or air to drive the transport of sample across the membrane and into the capturing chamber(s). An advantageous kind of material are porous materials from which all gas has been removed by application of vacuum which are stored as such, with no contact with air. Once such materials are exposed to the environment, they will immediately adsorb the closest material, such as air or liquid to restore balance or equilibrium. Thus, in the present use, such a material may be used for the tube to provide for the transport from the extraction point and up to, and through, the membrane of the device. This mechanism is for example utilised in Samba sensors. In one embodiment, the tube or capillary is made from silicone or a material with similar behaviour.

In one embodiment, the tube is a capillary, using capillary forces to transport a liquid sample.

The device according to the invention may be provided in a housing, comprising detector, in-put and out-put elements and other elements required for making it a suitable point-of-care product.

Thus, in one embodiment, the device is arranged in a housing which also comprises means for single particle counting of analyte-particle clusters at the downstream side of the membrane. In an alternative embodiment, the housing comprises means for other detection such as fluorescence detection of labelled particles forming clusters with analyte.

As the skilled person will appreciate, the particles could be labelled and detected using any conventional method, which will not interfere or be affected by the relevant analyte.

In a specific embodiment, the housing comprising the device according to the invention also comprises means for extracting a blood sample from an individual. A blood sample may comprise one or more analytes of interest, hence the device according to the invention may comprise more than one capturing chambers, one for each analyte. For example, if the housing comprising the device according to the invention is a point-of-care product for patients suffering from diabetes, the device may comprise analytes capable of detecting blood sugar levels and inflammation analytes. Alternatively, if the housing is a product for use in efficient physical exercise, it may comprise an analyte for lactic acid.

In a specific embodiment, the housing according to the invention is a disposable product. In an alternative embodiment, elements of the housing such as the capillary comprising capturing chamber(s), the needle for extracting a sample and/or the wash liquids are disposable while the sensing equipment, the display and/or the computer and software are for reuse with replaced consumables.

Thus, the device according to the present invention may be used for a large variety of applications, advantageously where sensitivity, precision, time to result and simplicity are important.

Consequently, a second aspect of the invention is a method of detecting at least one analyte in a liquid sample using a device comprising a tube in which a non-binding membrane has been arranged, which method comprises
a) Providing a liquid sample at an application region of a non-binding membrane arranged upstream of a capturing chamber wherein labelled particles have been arranged;
b) Passing the sample across the membrane and into the capturing chamber;
c) Allowing at least one analyte present in the sample to form clusters with the labelled particles in the capturing chamber;
d) Optionally, flushing a wash liquid through the capturing chamber and membrane in a direction opposite to the flow of step b) to remove unbound particles; and
e) Detecting clusters formed of labelled particles and analyte retained in the capturing chamber,
   wherein the cut-off of the membrane is large enough to allow passage of labelled particles but small enough to retain clusters comprised of analyte bound to particles.

In one embodiment, the method further comprises results from the detection into a value which provides information about the status of the individual from which the liquid sample originated.

In an advantageous embodiment, the method comprises a step of directly obtaining the liquid sample from an individual.

The particles as well as the materials of the device may be as discussed above in regard to the first aspect of the invention. As discussed above, two differently labelled particles directed to different binding sites of the same analyte may be arranged in each capturing chamber.

In one embodiment, the sample is selected from the group consisting of blood, plasma, saliva, sweat and urine. In an advantageous embodiment the sample is blood withdrawn using conventional needle technology, for example as commonly used for blood glucose testing.

The detection of step (d) may e.g. be by single particle counting.

The analyte may be a biomarker which is an indicator of a clinical condition, or the state of a specific clinical condition. Alternatively, the analyte is a biomarker providing any other health-related information, sometimes called a “health metric”.

Examples of such health metrices may be adrenalin, which indicates stress or stress levels, hormones, indicating a specific stage of a fertility cycle, lactic acid, indicating physical stress etc. As a further alternative, the biomarker may be an indicator which is not an indicator of a clinical condition, but rather indicates to an individual at risk that professional advice may be appropriate to seek—such as a biomarker indicating heart or similar conditions.

In one embodiment, the analyte is a biomolecule, such as a peptide or protein, or any molecule comprising a peptidic structure. In an alternative embodiment, the analyte is a nucleic acid or an organic molecule.

In one embodiment, the reagents specific to unique binding sites are antibodies, antibody fragments or fusion proteins comprising antibodies or fragments thereof. The technology for creating antibodies such as monoclonal antibodies directed to certain epitopes of proteins is by now well known to those of skill, and antibodies specific to defined epitopes or target biomarkers may be ordered and purchased from commercial suppliers.

The method according to the invention may use a device according to any one of the above-discussed embodiments. The skilled person may envisage further embodiments and/or combinations of embodiments based on this specification and drawings, which may not be discussed herein in detail but will fall within the scope of the appended claims.

Terms and expressions used in the present specification should be understood to have the conventional meaning applied by those skilled in the present technical area.

1-18. (canceled)

19. An analytical device comprising at least one non-binding membrane (1); at least one liquid sample application region; and at least one capturing chamber (3) comprising labelled particles (4); in which device the sample application region(s) are arranged upstream of the membrane while the capturing chamber(s) (3) are arranged downstream of the membrane, and wherein the cut-off of the membrane is large enough to allow passage of labelled particles but small enough to retain clusters comprised of analyte bound to particles.

20. A device according to claim 19, wherein the membrane is defined by having no lateral crossover between individual pores.

21. A device according to claim 19, wherein the capturing chamber(s) are arranged in a material (5′) from which air has been evacuated and air is prevented to re-enter.

22. A device according to claim 19, wherein the capturing chamber(s) are arranged in a material (5′) from which air has been evacuated, which evacuated material comprises silicone.

23. A device according to claim 19, wherein at least one capturing chamber (3) is provided with at least one wash liquid inlet (5) separated from the membrane.

24. A device according to claim 19, wherein the labelled particles (4) are comprised of DNA and/or RNA.

25. A device according to claim 19, wherein the labelled particles (4) comprise reagent(s) specific to an analyte.

26. A device according to claim 19, wherein the labelled particles (4) comprise reagent(s) specific to an analyte, which reagents are antibodies specific to at least one epitope of the analyte, and wherein two differently labelled antibodies directed to different epitopes of the same analyte are arranged in at least one capturing chamber (3).

27. A device according to claim 19, wherein the capturing chamber(s) are arranged in a tube (8).

28. A device according to claim 19, which is arranged in a housing which also comprises detecting means (6) for single particle counting of analyte-particle clusters in the capturing chamber(s) (3).

29. A device according to claim 19, which is arranged in a housing which also comprises detecting means (6) for single particle counting of analyte-particle clusters in the capturing chamber(s) (3), wherein the housing comprises means for extracting a blood sample (2′) from an individual.

30. A method of detecting at least one analyte in a liquid sample, which method comprises
a) Providing a liquid sample (2′) at an application region of a non-binding membrane (1) arranged upstream of a capturing chamber (3) wherein labelled particles (4) have been arranged;
b) Passing the sample across the membrane and into the capturing chamber (3);
c) Allowing at least one analyte present in the sample to form clusters with the labelled particles (4) in the capturing chamber;
d) Optionally, flushing a wash liquid through the capturing chamber (3) and membrane in a direction opposite to the flow of step b) to remove unbound particles; and
e) Detecting clusters formed of labelled particles (4) and analyte retained in the capturing chamber (3),
wherein the cut-off of the non-binding membrane (1) is large enough to allow passage of labelled particles (4) but small enough to retain clusters comprised of analyte bound to particles.

31. A method according to claim 30, wherein the capturing chamber (3) has been arranged in a material (5′) from which air has been evacuated, and step b) is provided by releasing the material from the vacuum.

32. A method according to claim 30, wherein two differently labelled particles (4) directed to different binding sites of the same analyte are arranged in the capturing chamber (3).

33. A method according to claim 30, wherein the liquid sample is selected from the group consisting of blood, plasma, saliva, sweat and urine.

34. A method according to claim 30, wherein the analyte is a peptide or protein.

35. A method according to claim 30, wherein the labelled particles comprise antibodies, antibody fragments and/or fusion proteins.

36. A method according to claim 30, wherein an analytical device comprising at least one non-binding membrane (1); at least one liquid sample application region; and at least one capturing chamber (3) comprising labelled particles (4); in which device the sample application region(s) are arranged upstream of the membrane while the capturing chamber(s) (3) are arranged downstream of the membrane, and wherein the cut-off of the membrane is large enough to allow passage of labelled particles but small enough to retain clusters comprised of analyte bound to particles.

37. The device of claim 25, wherein the reagent(s) specific to an analyte are antibodies specific to at least one epitope of the analyte.
